(19) United States
(12) Patent Application Publication
(10) Pub. No.: US 2009/0209516 A1
(43) Pub. Date: Aug. 20, 2009

(54) 9-AMINO-ACRIDINE DERIVATIVES AND METHOD OF TREATING AUTOIMMUNE DISEASES USING THE SAME

(76) Inventors: Carsten Korth, Dusseldorf (DE); Ralf Klingenstein, Hilden (DE); Stefan Lober, Ruckersdorf (DE); Peter Gmeiner, Erlangen-Buckenhof (DE); B. Kieseier, Meerbusch (DE); G. Meyer zu Horste, Burgdorf (DE); Olaf Stuve, Dallas, TX (US)

Correspondence Address: RANKIN, HILL & CLARK LLP, 38210 Glenn Avenue, WILLOUGHBY, OH, 44094-7808, US

(21) Appl. No.: 12/195,630

(22) Filed: Aug. 21, 2008

Related U.S. Application Data
(63) Continuation-in-part of application No. 11/817,622, filed on Mar. 20, 2008.
(60) Provisional application No. 60/969,381, filed on Aug. 31, 2007.

(30) Foreign Application Priority Data
Mar. 1, 2005 (DE) 102005009909.2

(51) Int. Cl. A61K 31/55; A61P 37/00

(52) U.S. Cl. 514/217

(57) ABSTRACT

The invention relates to compounds according to general formula (1) and/or their enantiomers, diastereomers, and their pharmaceutically compatible salts, and their use to manufacture a medication as well as medications. The compounds are suited for treatment of diseases connected with misfolded proteins.

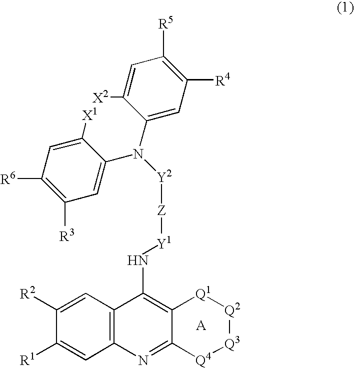

### RELATED APPLICATIONS

This application claims the benefit, under 35 U.S.C. §119(e), of U.S. Ser. No. 60/969,381, filed Aug. 31, 2007, and is a continuation-in-part of co-pending Application Ser. No. 11 /817,622, filed Aug. 31, 2007, which application is a 35 U.S.C. §371 (a) of International Application No. PCT/EP2006/060328, filed Feb. 28, 2006, and claims priority to DE 10 2005 009 909.2, filed Mar. 1, 2005.

### BACKGROUND OF THE INVENTION

1. Field of Invention

The invention relates to compounds, their enantiomers, diastereomers, and their pharmaceutically compatible salts, and their use to manufacture medications as well as medications. The compounds are suited for treatment of diseases connected with misfolded proteins and in the treatment of autoimmune diseases.

2. Description of Related Art

Prion diseases are fatal neurodegenerative diseases that can appear both in humans, for example Creutzfeld-Jacob disease (CJD), and in the animal realm, for example bovine spongiform encephalopathy (BSE) in cattle, or scrapie in sheep. The infectious agent is so-called prion, which consists of a misfolded form of a protein that is present in the organism, the prion protein. In cases of disease, this prion accumulates in the central nervous system and there leads to cell death. Although these diseases rarely occur in humans, public focus has in recent years been directed to them, since it has been shown that BSE present in cattle can be transferred via the food chain to humans and can trigger CJD there.

Extracellular or intracellular protein aggregations are also a phenotypic sign of chronic degenerative diseases, especially neurodegenerative and/or neuropsychiatric diseases, for example Alzheimer's, Parkinson's or tauopathies.

In the state of the art, various substances are known that show effectiveness against prions. However, what is disadvantageous in the known substances is that due to their polarity, they cannot overcome the blood-brain barrier and thus cannot get into the brain. Moreover, many of the substances are not effective in low doses, but reveal a toxic effect at higher dosages.

The acridine and phenothiazine derivatives published in WO 02/096431, for example, reveal a disadvantage in that at low dosages, they are not effective or only slightly so. Additionally, acridine, especially bis-acridine, can have toxic effect. Also, these substances reveal merely a transient improvement in the disease symptoms, but no long-lasting therapeutic effect. This reveals a further disadvantage of the known substances, which manifest no effect in the stages of advanced or clinical disease.

### BRIEF SUMMARY OF THE INVENTION

The task of the present invention was to make available compounds that overcome at least one of the disadvantages of the state of the art. Particularly the task of the invention was to make compounds available that exhibit improved effectiveness against chronic degenerative diseases.

This and other problems are solved through the use of compounds according to the overall formula (1) as indicated in what follows, and/or their enantiomers, diastereomers and their pharmaceutically compatible salts:

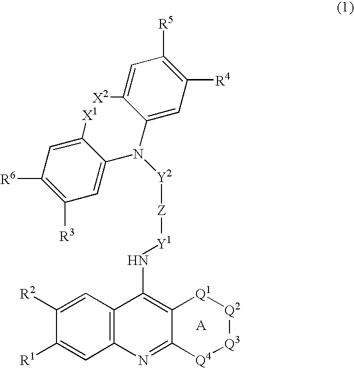

wherein:
A is a six-link, unsaturated or saturated ring;
Q¹, Q², Q³, Q⁴ are each selected, independent of each other, from the group including CH, C-halogen, C—O—(C₁-C₁₀)-alkyl, C—CF₃, C—CN and/or CH₂;
R¹, R², R³, R⁴, R⁵, R⁶, are each selected, independent of each other, from the group including H, Halogen, C₁-C₁₀-alkyl, C₁-C₁₀-alkenyl, C₁-C₁₀-alkinyl, C₁-C₁₀-alkyloxy, CF₃, NH₂, NHR⁹, whereby the radical R⁹, is selected from the group including C₁-C₁₀-alkyl and/or C₁-C₁₀-acyl, NO₂, and/or CN;
X¹, X² each are H, or X¹ and X² jointly form X, whereby:
X is selected from the group including CH₂, CH₂—CH₂, CH═CH, O and/or S;
Y¹, Y² each, independent from each other, are unbranched or branched C₁-C₁₀-alkyl;
Z is a structure element Z1 or Z2 as given above, whereby:
Z1 has the following general formula (2):

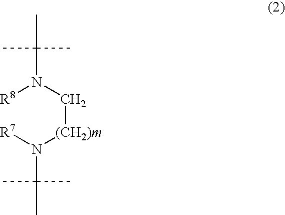

wherein:
m is 1, 2, 3, 4, 5 or 6,
R⁷, R⁸, are selected independently of each other from the group including H, C₁-C₁₀— alkyl, whereby R⁷ and R⁸, if necessary via a CH₂—CH₂ group, form a ring, and/or C₁-C₁₀-acyl; and
Z2 has the following general formula (3):

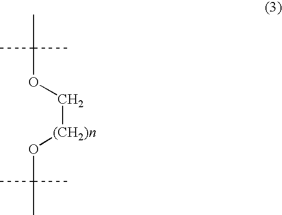

wherein
n is 2, 3, 4, 5 or 6.

Surprisingly, it was found that the invention-specific compounds can get through cell membranes.

A particular advantage of the compounds is hereby obtained, in that these compounds can be used in lower doses, which makes it possible to use the compounds particularly in humans.

Of particular advantage is that the invention-specific compounds exhibit reduced toxicity.

Surprisingly, it was further found that the invention-specific compounds can have a positive effect on misfolding of proteins.

Especially surprisingly, it was found that the secondary amino group in the side chain of the invention-specific compounds according to formula (1) can significantly increase the effectiveness compared to a tertiary amino group.

In addition, applicants have surprisingly discovered that such compounds are useful in treating autoimmune diseases in humans and animals

### BRIEF DESCRIPTION OF THE DRAWINGS

FIG. 1 shows Western blots of cell lysates of untreated cells, cells treated with Quinacrin and cells treated with quinapramine (sometimes hereinafter referred to as “PAC05”), which is a compound according to the invention.

FIG. 2 is a composite of six graphs labeled A, B, C, D, E and F, respectively, which shows the results of various experiments in which mice were treated PAC05.

FIG. 3 is a composite of four graphs labeled A, B, C and D, respectively, which shows the results of PAC05 therapy on antigen-specific T cell proliferation and on cytokine expression of antigen-specific splenocytes in mice.

FIG. 4 is Clinical EAN score of Lewis rats treated with PAC05 and vehicle from day 11 until day 38 post immunization with bovine peripheral nerve homogenates.

### DETAILED DESCRIPTION OF THE INVENTION

The present invention provides compounds according to the overall formula (1) as indicated below and in what follows, and/or their enantiomers, diastereomers and their pharmaceutically compatible salts:

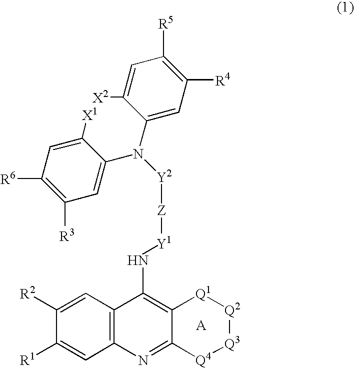

The structural element A is a six-link, unsaturated or partially saturated ring, whereby A is a carbon ring. Q¹, Q², Q³, Q⁴ include corresponding carbon atoms that form a carbon ring. The structural elements Q¹, Q², Q³, and Q⁴ can, in correspondence to the saturation of the A ring, form groups CH or CH₂.

The structural elements Q¹, Q², Q³, and Q⁴ can further have substituents on the carbon atoms, whereby the substituents are selected from the group that includes H, Halogen, C₁-C₁₀-alkyloxy, CF₃ and/or CN. Preferred substituents are selected, independent of each other, from the group including H, Halogen from the group including F, Cl and/or Br, and/or C₁-C₅ alkyloxy, preferably selected from the group including —O-methyl, —O-ethyl, —O-isopropyl and/or —O-tert-butyl.

The structural elements Q¹, Q², Q³, and Q⁴ can be appropriately selected independent of each other from the group including CH, CH₂, C-halogen, C—O—(C₁-C₁₀)-alkyl, C—CF₃ and/or C—CN. Preferably the structural elements Q¹, Q², Q³, and Q⁴ are selected independent of each other from the group including CH, C-halogen and/or C—O—(C₁-C₁₀)-alkyl, C—O-isopropyl and/or C—O-tert-butyl. Especially preferred are the structural elements Q¹, Q², Q³, and Q⁴ selected independent of each other from the group including Ch, C—Cl and/or C—O-methyl.

In preferred embodiment form, the structural element A is a six-link, unsaturated carbon ring. Additionally, the structural element Q² preferably has substituents. Preferably the structural elements Q¹, Q³, and Q⁴ each form a group CH and the structural element Q² is selected from the group including CH and/or C—O—CH₃.

In further preferred embodiment forms the structural element A is a six-link, partially saturated carbon ring, especially preferred a non substituted six-link, partially saturated ring. In these embodiment forms, preferably all the structural elements Q¹, Q², Q³, and Q⁴ form a CH₂ group.

The radicals R¹, R², R³, R⁴, R⁵, and R⁶ from the invention-specific compounds are each selected, independently of each other, from the group including H, halogen, C₁-C₁₀-alkyl, C₁-C₁₀-alkenyl, C₁-C₁₀-alkinyl, C₁-C₁₀-alkyloxy, CF₃, NH₂, NHR⁹, whereby the radical R⁹, is selected from the group including C₁-C₁₀-alkyl and/or C₁-C₁₀-acyl, NO₂, and/or CN.

Preferably, the radicals R¹, R², R³, R⁴, R⁵, and R⁶ are each selected, independent of each other, from the group including H, halogen, selected from the group including F, Cl and/or Br, NH₂, NHR⁹, whereby the radical R⁹, is selected from the group including C₁-C₁₀-alkyl, preferably C₁-C₅-alkyl, preferably selected from the group including methyl, ethyl, isopropyl and/or tert-butyl, and/or C₁-C₁₀-acyl, preferably C₁-C₅-acyl, NO₂, C₁-C₁₀-alkyl, preferably selected from the group including methyl, ethyl, n-propyl, isopropyl, n-butyl, tert-butyl, n-pentyl, n-hexyl, n-heptyl and/or n-octyl, preferably C₁-C₅-alkyl, preferably selected from the group including methyl, ethyl, isopropyl and/or —O-tert-butyl. In preferred embodiment forms of the invention-specific compounds, the radicals R¹, R², R³, R⁴, R⁵, and R⁶ are each selected, independent of each other, from the group including H, Cl and/or —O-methyl, especially preferred, selected independent of each other from the group including H and/or Cl.

A substitution with groups R¹, R², R³, R⁴, R⁵, and R⁶ which are each selected independent of the other from the group including H and/or Cl, can result in a considerable increase in the effectiveness of the compound.

The structural elements X¹ and X² can each be H, or jointly form a structural element X. Embodiment forms including structural elements X¹ and X² each of which are H, can, for example, have a part structure according to the following formula (4):

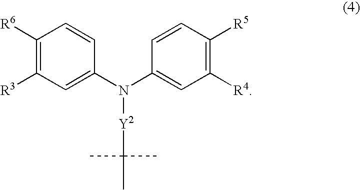

If structural elements X¹ and X² jointly form a structural element X, a part structure according to the following formula (5) is produced:

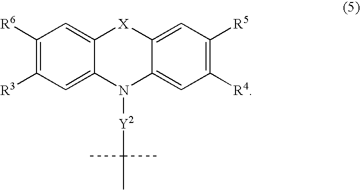

The structural element X can be a heteroatom, for example O or S, or be selected from the group including CH₂, CH₂—CH₂, and/or CH═CH. Preferably ring systems result, that have a central 6- or 7-link ring.

Especially preferred invention-specific compounds have a structural element X selected from the group including S and/or —CH₂—CH₂—.

Preferably the invention-specific compounds include a structural element Z1. Preferred embodiment forms including a structural element Z1 have the following formula (6):

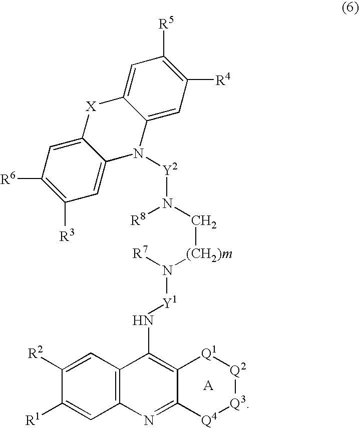

The radicals R⁷ and R⁸ are preferably H or alkyl groups with C₁ to C₁₀ like methyl, ethyl, n-propyl, isopropyl, n-butyl, tert-butyl, n-pentyl, n-hexyl, n-heptyl and/or n-octyl, preferably C₁-C₅-alkyl, especially selected from the group including methyl, ethyl, isopropyl and/or tert-butyl. In these embodiment forms, m preferably is 1, 2, 3, 4, 5 or 6.

Preferably the structural element Z1 has a ring-shaped structure, whereby the ring closes via the radicals R⁷ and R⁸. Preferably the ring is formed by each of the radicals R⁷ and R⁸ forming a CH₂ group, and jointly forming a CH₂—CH₂ group. In these embodiment forms, m is preferably 1 or 2, so that a heterocyclic 6- or 7-link ring is formed.

In preferred embodiment forms, m is equal to 1 and the structural element Z1 includes a piperazine heterocycle. The structural element Z1 correspondingly has the following formula (7) in preferred embodiment forms:

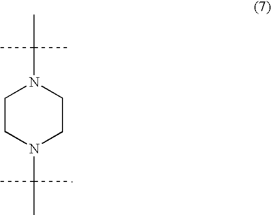

Additional preferred embodiment forms including a structural element Z2 have the following formula (8), whereby n preferably is 2, 3, 4 or 5:

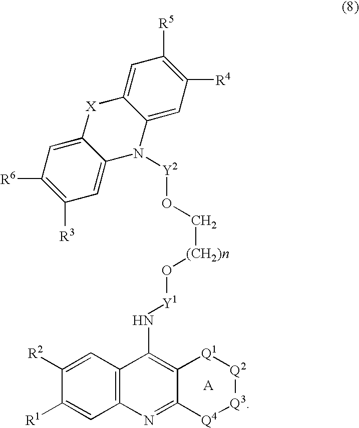

The structural elements Y¹, Y² are each, independent from each other, an unbranched or branched C₁-C₁₀-alkyl.

Especially preferred compounds and/or their enantiomers, diastereomers as well as their pharmaceutically compatible salts, have the overall formula (9), as given next:

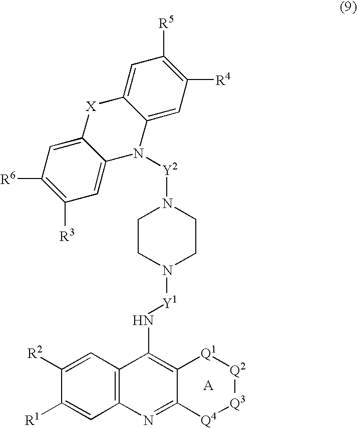

wherein:
A is a six-link, unsaturated or saturated ring;
Q¹, Q², Q³, Q⁴ are each selected, independent of each other, from the group including CH, C-halogen, C—O—(C₁-C₁₀)-alkyl, C—CF₃, C—CN and/or CH₂;
R¹, R², R³, R⁴, R⁵, R⁶ are each selected, independent of each other, from the group including H, Halogen, C₁-C₁₀-alkyl, C₁-C₁₀-alkenyl, C₁-C₁₀-alkinyl, C₁-C₁₀-alkyloxy, CF₃, NH₂, NHR⁹, whereby the radical R⁹, is selected from the group including C₁-C₁₀-alkyl and/or C₁-C₁₀-acyl, NO₂, and/or CN;
X is selected from the group including CH₂, CH₂—CH₂, CH═CH, O and/or S; and
Y¹, Y² each, independent from each other, are unbranched or branched C₁-C₁₀-alkyl.

Preferably the structural elements Q¹, Q², Q³, and Q⁴ of the invention-specific compounds each form a CH group and the structural element Q² is selected from the group including CH and/or C—O—CH₃. In especially preferred embodiment forms, the structural elements Q¹, Q³, and Q⁴ each form a CH₂ group, and the structural element Q² is C—O—CH₃. In further preferred embodiment forms, preferably all the structural elements Q¹, Q², Q³, and Q⁴ form a CH₂ group.

Preferably, the radicals R¹, R², R³, R⁴, R⁵, and R⁶ are each selected, independent of each other, from the group including H, halogen, selected from the group including F, Cl and/or Br, NH₂, NHR⁹, whereby the radical R⁹, is preferably selected from the group including C₁-C₅-alkyl, preferably selected from the group including methyl, ethyl, isopropyl and/or tert-butyl, and/or C₁-C₁₀-acyl, preferably C₁-C₅-acyl, NO₂, C₁-C₅-alkyl, preferably selected from the group including methyl, ethyl, isopropyl, and/or tert-butyl, and/or C₁-C₅-alkyloxy, preferably selected from the group including —O-methyl, —O-ethyl, —O-isopropyl and/or —O-tert-butyl. In preferred embodiment forms of the invention-specific compounds, the radicals R¹, R², R³, R⁴, R⁵, and R⁶ are each selected, independent of each other, from the group including H, Cl and/or —O-methyl, especially preferred, selected independent of each other from the group including H and/or Cl.

Especially preferred compounds have a structural element X selected from the group including S and/or CH₂—CH₂.

The structural elements Y¹ and Y² of the invention-specific compounds can each, independent of the other, have at least one branching, whereby methyl and/or ethyl side chains are preferred. Preferably, the structural elements Y¹ and Y² are each selected, independent of each other, from the group including —(CH₂)ₒ—, whereby o is 2, 3, 4, 5 or 6, preferably 3 or 4, and/or —CH(CH₃)— (CH₂)ₚ—, whereby p is 2, 3, 4, or 6, preferably 3 or 5, especially preferred 3. In very particularly preferred embodiment forms, the structural elements Y¹ and Y² are each, independent of each other, —(CH₂)ₒ—, whereby o is 3, 4 or 5.

In especially preferred compounds, the distance formed by the group of structural elements Y¹-Z-Y² between each of the nitrogen atoms connecting to the structural elements Y¹ and Y² is a length from 10 Å to 15 Å.

Especially preferred compounds have the structural elements Q¹, Q³, and Q⁴ each of which forms a CH₂ group, and a structural element Q² selected from the group including CH and/or C—O—CH₃. whereby the structural element Q² preferably is C—O—CH₃, and structural elements Y¹ and Y² which, independent of each other, are each selected from the group including —(CH₂)ₒ— and/or —CH(CH₃)— (CH₂)ₚ—, whereby o is 2, 3, 4, 5 or 6, and p is 2, 3, 4 or 5.

Additional preferred compounds have the structural elements Q¹, Q³, and Q⁴ each of which forms a CH₂ group, and a structural element Q² selected from the group including CH and/or C—O—CH₃, whereby the structural element Q² preferably is C—O—CH₃, and structural elements Y¹ and Y² which, independent of each other, are —(CH₂)ₒ—, whereby o is 3, 4, or 5.

Very particularly suitable compounds include structural elements Q¹, Q³, and Q⁴ each of which forms a CH₂ group, and a structural element Q² selected from the group including CH and/or C—O—CH₃, whereby the structural element Q² preferably is C—O—CH₃, and structural elements Y¹ and Y² which, independent of each other, are —(CH₂)ₒ—, whereby o is 3, 4, or 5, and the radicals R¹, R², R³, R⁴, R⁵, and R⁶, each selected, independent of the other, from the group including H and/or Cl.

Especially preferred embodiment forms of the invention-specific compounds have the formula (10) given as follows, and/or are their enantiomers, diastereomers as well as their pharmaceutically compatible salts:

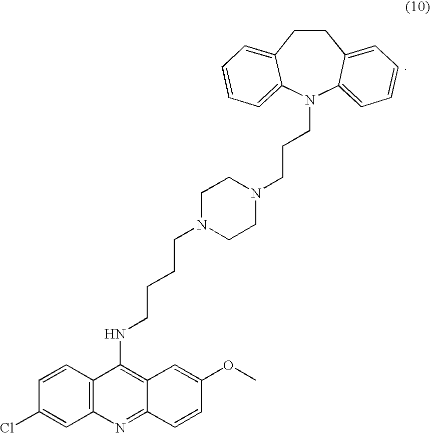

Such compounds are sometimes referred to herein as quinpramine or simply as “PAC05”. The molecule according to formula (1) comprises both an acridine (quinacrine) and an iminodibenzyl (imipramine) moiety. It is noteworthy that the “parent” compounds of PAC05, namely quinacrine and imipramine, are both approved drugs with pharmaceutically acceptable side effects and toxicity profiles, potentially facilitating a corresponding profiling of side effects in humans.

Further preferred embodiment forms of the invention-specific compounds have the formula (11) given next, and/or are their enantiomers, diastereomers as well as their pharmaceutically compatible salts:

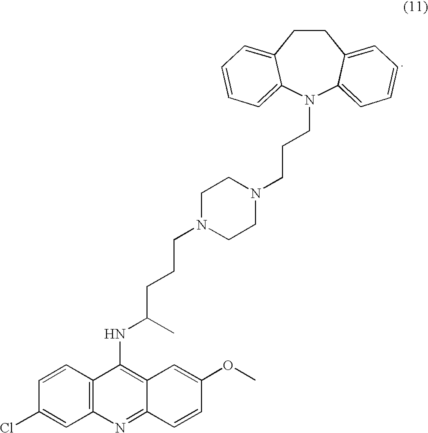

Also preferred embodiment forms of the invention-specific compounds have the formula (12) given next, and/or are their enantiomers, diastereomers as well as their pharmaceutically compatible salts:

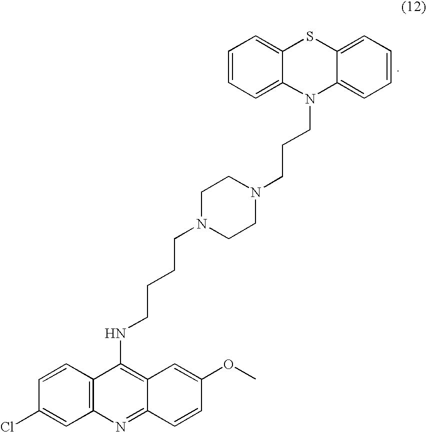

Also preferred embodiment forms of the invention-specific compounds have the formula (13) given next, and/or are their enantiomers, diastereomers as well as their pharmaceutically compatible salts:

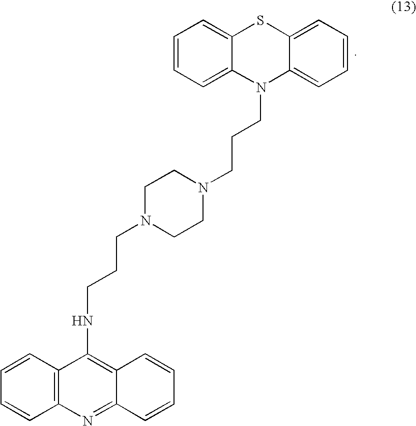

Also preferred embodiment forms of the invention-specific compounds have the formula (14) given next, and/or are their enantiomers, diastereomers as well as their pharmaceutically compatible salts:

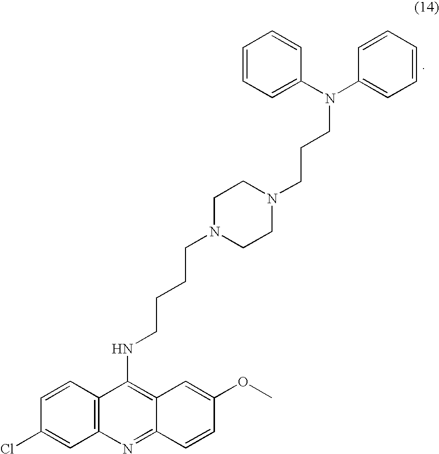

Preferred compounds are selected from the group including (6-chlor-2-methoxy-acridine-9-yl)-(4-{4-[3-(10,11-dihydro-dibenzo[b,f]azepine-5-yl)-propyl]-piperazine-1-yl}-butyl)-amine and/or (6-chlor-2-methoxy-acridine-9-yl)-(3-{4-[3-(dihydro-dibenzo[b,f]azepine-5-yl)-propyl]-piperazine-1-yl}-butyl)-amine, whereby the first-name corresponds to compound (10). As noted, this compound is sometimes referred to herein as “quinpramine” or simply as “PAC05”.

Further preferred embodiment forms of the invention-specific compounds have the formulas (15) to (24) given next, and/or are their enantiomers, diastereomers as well as their pharmaceutically compatible salts:

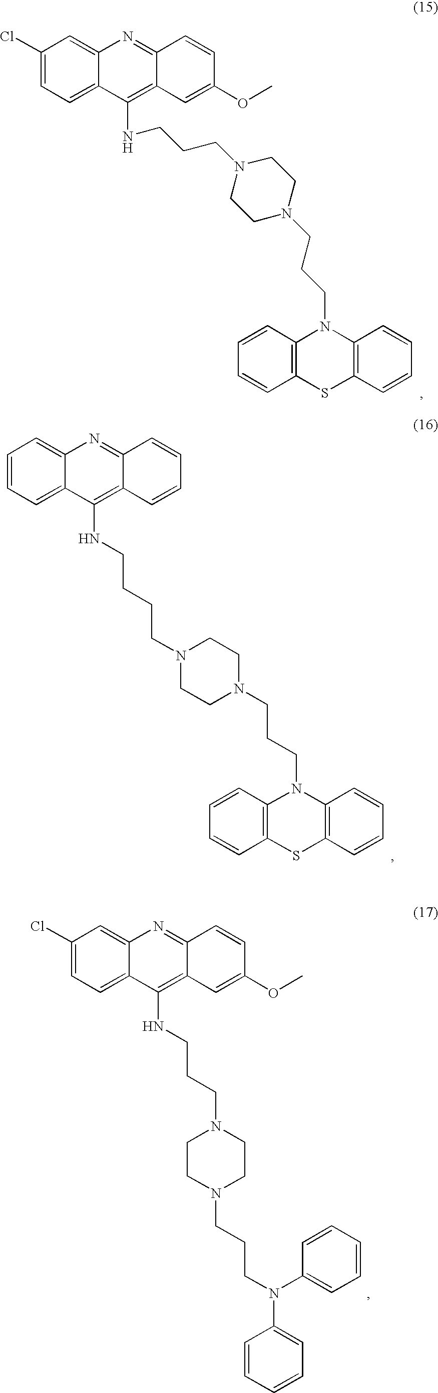

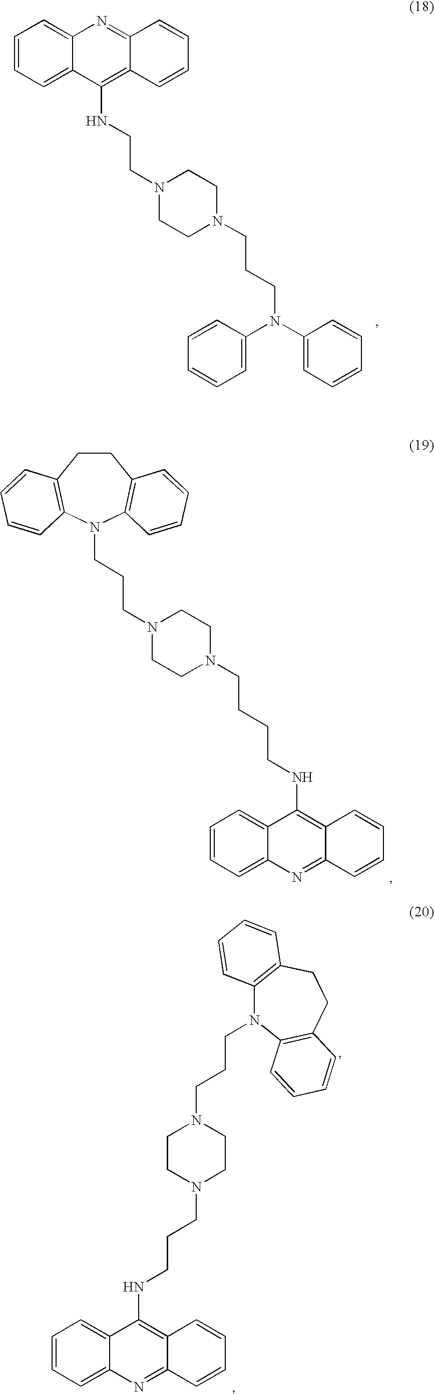

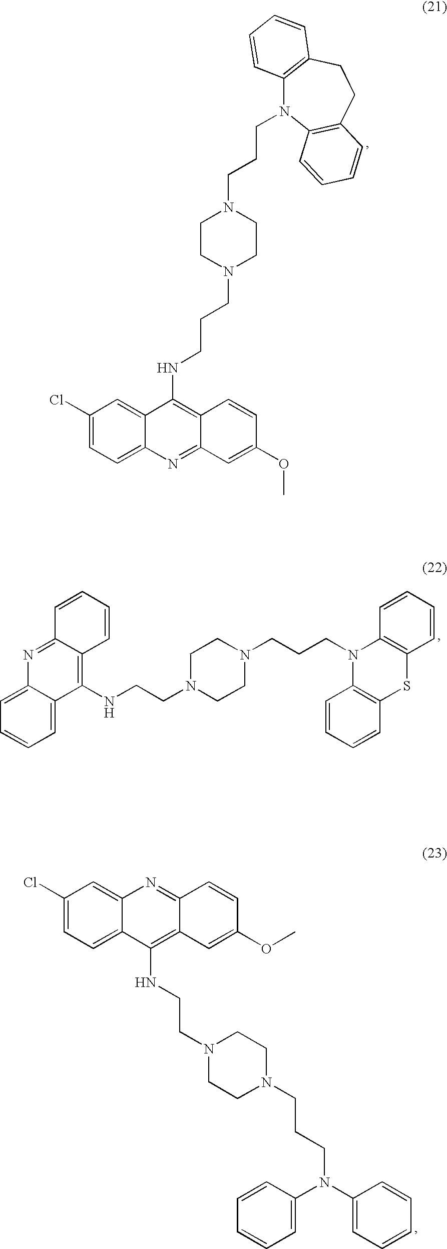

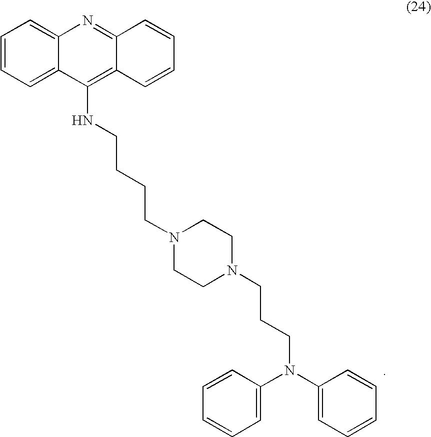

The compounds can be manufactured according to the customary synthesis methods.

One advantage of the compounds lies in preferred embodiment forms in that these compounds can get through cell membranes. In particular, the invention-specific compounds have good ability to pass the blood-brain barrier. This makes it possible for the invention-specific compounds to be used in vivo and in vitro. Invention-specific compounds can be used, for example, in cell cultures. In especially preferred embodiment forms, invention-specific compounds are also usable in tissues or organs. Particularly, the invention-specific compounds make it possible to use them in organisms.

Due to their advantageous properties, the invention-specific compounds are suitable for use as medications.

A further subject of the invention is the use of the invention-specific compounds, particularly compounds with the overall formulas (10) to (14), to produce a medication.

The invention-specific compounds can be administered by the customary methods, with oral or dermal administration being preferred, and/or administration by injection, such as intravenously, subcutaneously and/or intramuscularly. Oral administration is especially preferred.

One advantage of the invention-specific compounds is that they have improved ability to pass the blood-brain barrier. Due to this, the compounds are highly effective at their activity site, particularly in the brain cells. What is meant by “passing the blood-brain barrier” in the context of the invention is that the compounds can penetrate through the blood-brain barrier and into the brain, into the brain cells, especially into neurons. In especially advantageous embodiment forms, all or nearly all of the compounds reach the brain cells, and/or are fully or nearly fully available in the brain cells.

This is especially advantageous, since the amount of the compounds to be administered with the invention-specific compounds can be considerably smaller than with substances that manifest less ability to pass the blood-brain barrier. In advantageous fashion, side effects can be reduced by administering smaller doses.

Preferred dosages of the invention-specific compounds for administration to humans are in the area from ≧1 mg per day per 75 kg of body weight to ≦1000 mg per day per 75 kg of body weight, advantageously in the area from ≧10 mg per day per 75 kg of body weight to ≦500 mg per day per 75 kg of body weight, preferably in the area from ≧30 mg per day per 75 kg of body weight to ≦100 mg per day per 75 kg of body weight, and especially preferred in the area of >30 mg per day per 75 kg of body weight to <50 mg per day per 75 kg of body weight.

The invention-specific compounds are usable in advantageous embodiment forms, especially for therapeutic and/or prophylacic treatment, diagnosis and/or therapy for diseases that are connected with misfolding of proteins. Among these diseases, for example, are prion diseases and/or chronic degenerative diseases, especially neurodegenerative and/or neuropsychiatric diseases.

Under the term “misfolding of proteins,” within the context of the invention, what is understood is that proteins may be misfolded or incorrectly processed.

In advantageous fashion, the invention-specific compounds can be used to treat plants and/or animals, tissues and/or cells. Especially the invention-specific compounds can be used to treat mammals such as humans.

The invention-specific compounds can have a positive influence on misfolding of proteins. Especially it was a surprise to find that preferred embodiment forms of the compounds can reduce the amount of misfolded prion proteins in cell culture models. In advantageous fashion, the invention-specific compounds reveal a higher effectiveness in influencing a misfolding of proteins than compounds known to date. Especially the invention-specific proteins can also demonstrate a greater effectiveness than combinations of substances known to date.

One particular advantage of the invention-specific compounds can be implemented in that the time from an infection to outbreak of the disease can be prolonged through administration of the invention-specific compounds, and/or the survival time after outbreak of the disease can be prolonged.

It is especially advantageous that the invention-specific compounds can lead to a reduction in misfolded proteins, especially prion proteins, even when the disease is advanced or clinical.

Experiments revealed that the invention-specific compounds (i.s.c.) reduce the cholesterol concentration on the cell surface. From this, applications also result for the i.s.c. to disturbances in the lipid-cholesterol metabolism. Additionally, an anti-parasitic-anti-protozoan effect is known from the acridines and phenothiazines. This effect is likewise attained by the i.s.c. and the result of this is applications to therapy of protozoa and parasitic diseases. We especially suspect that the invention-specific compounds may demonstrate a positive effect in treatment of trypanolysis.

Without being committed to a specific theory, we further deduce that the invention-specific compounds can have a positive effect in the treatment and/or prophylaxis of Alzheimer's disease, and especially can demonstrate a positive effect on prevention of Alzheimer's disease.

Correspondingly, a further subject of the invention relates to use of the invention-specific compounds, primarily of the overall formulas (10) to (14), to produce a medication for therapeutic and/or prophylactic treatment of diseases, selected from the group including:
diseases that are connected with misfolding of proteins, like prion diseases and/or chronic degenerative diseases, especially neurodegenerative and/or neuropsychiatric diseases,
diseases connected with increased cholesterol in the blood,
diseases connected with a disturbance in the lipid metabolism, and/or hyperlipidemia,
diseases connected with an inflammation, like chronic inflammations or autoimmune diseases,
cardiovascular diseases, especially arteriosclerosis, and/or
infections in animals and plants evoked by parasites, especially in humans and commercially useful animals, diseases caused by protozoa and/or worms, especially cerebral infections caused by parasites and/or protozoa.

The prion diseases can especially be selected from the group including Creutzfeld-Jakob Disease, Gerstmann-Sträussler-Scheinker Disease, Fatal Familial Insomnia (FFI), kudu, scrapie and/or bovine spongiform encephalopathy (BSE).

Chronic degenerative diseases, particularly neurodegenerative and/or neuropsychiatric diseases, have preferably been selected from the group including Alzheimer's, amyotrophic lateral sclerosis (ALS), Pick's Disease, Parkinson's, multiple sclerosis, Huntington's chorea, type I and type II diabetes, autism, schizophrenia, bipolar diseases, depression, polyglutamine diseases, fronto-temporal dementia, tauopathies, multiple system atrophy, sleep disturbances, plasma cell dyscrasia, familial amyloid polyneuropathy, medullar thyroid carcinomas, chronic renal insufficiency, congestive heart failure, amyloidoses like cardiac amyloidosis, systemic amyloidosis and/or familial amyloidosis.

Preferred neurodegenerative and/or neuropsychiatric diseases are selected from the group including Alzheimer's, fronto-temporal dementia, Parkinson's, tauopathies, multiple system atrophy, amotrophic lateral sclerosis (ALS), schizophrenia, bipolar diseases, depression, polyglutamine diseases, multiple sclerosis and/or sleep disturbances.

Especially in treatment of neurodegenerative diseases in humans an advantageous effect can be attained on the course of the illness by use of the invention-specific compounds, and in particular the appearance of misfolded proteins can be reduced. A further advantage of the invention-specific compounds can result from the fact that they can be used in low doses. Additionally, the result of this is that no side effects, or only slight ones, appear. This makes it possible to administer the invention-specific compounds over a longer period of time. This, for example, makes possible administration for treatment of neurodegenerative diseases, therapy for which must under certain circumstances continue for months or years.

In especially advantageous embodiment forms, the invention-specific compounds can lead to a lowering of blood cholesterol. This, for example, makes possible an application for disturbances in lipid metabolism or hyperlipidemia. Additionally, the compounds can manifest anti-inflammatory effects and be used for therapeutic and/or prophylactic treatment of cardiovascular diseases, especially for prophylaxis of arteriosclerosis.

Additionally, the compounds are usable in advantageous fashion also for therapeutic and/or prophylactic treatment of malaria and other parasite-caused infections in living creatures, animals and plants, especially commercially useful animals and humans, such as malaria, trypanosomiasis, sleeping sickness, amoebiasis, leishmaniosis and/or toxoplasmosis. Also, application as an anti-protozoan agent and/or as an anti-helminthic, for example, against nematodes like pinworms (enterobius), ascaris, ascaris in dogs and cats (toxocara), whipworm (trichuris), hookworm (nacator, anchylostoma), strongyloid threadworm (spobngyloides), guinea worm (dracunculus), or filarial, zestodes, and/or trematodes.

One great advantage in using the invention-specific compounds, preferably of the formula (10) can be implemented in that they possess better ability to pass the blood-brain barrier, and can demonstrate better effectiveness with cerebral parasites and/or protozoan infections like cerebral malaria, toxoplasmosis and/or cerebral abcesses.

Without being bound to a specific theory, we further suspect that the invention-specific compounds, preferably of formula (10), can demonstrate an inhibiting effect on so-called multi-drug resistance (MDR) proteins, like the P-glycoprotein, so that better effectiveness and bioavailability can be offered in comparison to known substances.

In advantageous fashion, the invention-specific compounds exhibit slight, or negligible, toxicity when administered. This, for example, makes possible administration in high doses, especially one-time and/or multiple, temporally limited administration in dosages in the area of ≧10 mg per day per 75 kg of body weight up to ≦1000 mg per day per 75 kg of body weight, for example over a time period of at least three months. The slight or negligible toxicity of the invention-specific compounds also makes possible administration in so-called pulsed therapy. With this, doses in the area of ≧10 mg per day per 75 kg of body weight up to ≦1000 mg per day per 75 kg of body weight can, for example, be administered for one week, preferably at least twice for a week, interrupted by a period with no administration. Such an administration can further improve the positive effect of the compounds on misfolded proteins.

A further subject of the invention relates to medications including invention-specific compounds, preferably including compounds of formulas (10) to (14).

Medications including invention-specific compounds are applicable for treatment in vivo and in vitro. One preferred use of the medications including invention-specific compounds is therapeutic and/or prophylactic treatment of diseases connected with misfolding of proteins, like prion diseases and/or chronic degenerative diseases, especially neurodegenerative and/or neuropsychiatric diseases.

A further subject of the invention relates to anti-prion agents, including invention-specific compounds, preferably compounds of the formula (10).

The term “anti-prion agents” in the context of this invention has the meaning that the agent including invention-specific compounds can positively influence prion diseases. Especially, the quantity of misfolded prion proteins (PrP^{Sc}) can be reduced.

In preferred embodiment forms, use of the invention-specific compounds can result in a dropoff in the quantity of misfolded proteins, especially prion proteins (PrP^{Sc}), in the investigated media, cells, tissues and/or organs, and/or the appearance of misfolded proteins can be totally or almost totally avoided.

For example, in experiments, the invention-specific compounds reveal themselves to be more active as compared with known substances or combinations of them. Thus, the invention-specific compounds can cause the quantity of misfolded prion proteins to decline to a greater extent than with administration of individual known substances or combinations of them.

A further subject relates to the use of invention-specific compounds, preferably of compounds of the overall formula (10), for purification of biological fluids, especially body fluids that contain misfolded proteins, preferably prions, especially from cell suspensions. The invention-specific compounds can especially be used for purification of biological fluids contaminated with misfolded proteins, preferably prions.

The term “purification” in the context of this invention has the meaning that in biological fluids, the quantity of misfolded proteins is reduced after a dosage of the invention-specific compounds, and especially the term “purification” in the context of this invention can be understood in the meaning of the terms “clearing” or “clearance.” Especially, the invention-specific compounds, in particularly advantageous embodiment forms, can lead to a complete or almost complete elimination of the misfolded proteins.

The cell suspensions can, for example, be cerebrospinal fluid, preferably the cell suspensions are selected from the group including blood and/or or blood products.

For this, the fluids can be removed from the organism and the invention-specific compounds can be added outside the organism, or the invention-specific compounds can be added to the fluids without these being removed from the organism.

Yet another subject of the invention relates to procedures for purification of biological fluids that contain misfolded proteins, preferably prions, whereby invention-specific compounds, preferably of the formulas (10) to (14) are added to the biological fluid to be purified, especially cell suspensions like blood or blood products. Optionally, the compounds can again be separated from the biological fluid to be purified by, for example, chromatography, dialysis and/or adsorption, after an incubation.

In further advantageous embodiment forms of the procedure, the biological fluid, before and after the dosing of invention-specific compounds, can be tested using suitable methods for the quantity of misfolded proteins contained.

In advantageous embodiment forms of the procedure, the biological fluid to be purified, especially cell suspensions like blood or blood products, can first be removed from the organism, for example by punction, and after purification be returned to the organism, for example by plasmapheresis.

In especially advantageous embodiment forms of the procedure for purification of biological fluids like cell suspensions, especially blood, this includes the following steps:
a) optional removal of the biological fluid from an organism, for example by puncture;
b) incubation of the biological fluid with the invention-specific compounds;
c) optional separation of the compounds, for example by chromatography, dialysis and/or adsorption;
d) optional re-insertion of the biological fluid, for example by plasmapheresis.

Alternatively, for example, the fluids can be stored in the form of stored blood and/or placed back at a later time into the organism from which they were removed.

Applicants have also surprisingly discovered that compounds according to the invention, and particularly PAC05, may be useful in treating autoimmune diseases in humans and animals such as, for example: acute disseminated encephalomyelitis (ADEM); Addison's disease; ankylosing spondylitis; antiphospholipid antibody syndrome (APS); aplastic anemia; autoimmune dermatitis; autoimmune glomerulonephritis; autoimmune hepatitis; autoimmune neuritis; autoimmune vasculitis; autoimmune Oophoritis; autoimmune hemolytic anemia; chronic fatigue syndrome; coeliac disease; colitis ulcerosa; Crohn's disease; dermatomyositis; diabetes mellitus type 1; gestational pemphigoid; Goodpasture's syndrome; Graves' disease; Guillain-Barré syndrome (GBS); Hashimoto's disease; idiopathic thrombocytopenic purpura; Kawasaki's disease; lupus erythematosus; multiple sclerosis; myasthenia gravis; opsoclonus myoclonus syndrome (OMS); optic neuritis; Ord's thyroiditis; panarteritis nodosa; pemphigus; pernicious anemia; polyarthritis in dogs; polymyositis; primary biliary cirrhosis; progressive systemic sclerosis; progressive aplastic anemia; psoriasis; rheumatoid arthritis; Reiter's syndrome; sclerodermia; Sharp syndrome; Sjögren's syndrome; Takayasu's arteritis; temporal arteritis (also known as “giant cell arteritis”); warm autoimmune hemolytic anemia; and Wegener's granulomatosis.

PAC05 was formulated after findings that antiprion effects of quinacrine and imipramine had an additive effect stronger than the sum of their single effects, i.e. they were synergistic (Klingenstein et al., 2006). The one-molecule chimeric component comprised both an acridine (quinacrine) and an iminodibenzyl (imipramine) moiety, connected through variable linkers (Dollinger et al., 2006). For antiprion effects, a ca. 5-fold stronger antiprion effect was observed in a cell culture model of prion disease. The mechanism of action of PAC05 was discovered to be a destabilization of detergent-resistant membrane compartments, or “lipid rafts” (Klingenstein et al., 2006; unpublished observations).

Continuing these experiments in vivo, we discovered that treatment of wild-type mice with 1 mg/mouse/day of PAC05 orally (gavage) over 10 weeks did not generate obvious acute or chronic toxic effects. When mice were inoculated with prions 25 days prior to administrating 1 mg/mouse/day PAC05 orally, treated mice survived on average 10% longer than untreated animals.

The assumed mechanism of action (destabilization of lipid rafts) prompted us to investigate PAC05's effect on other diseases.

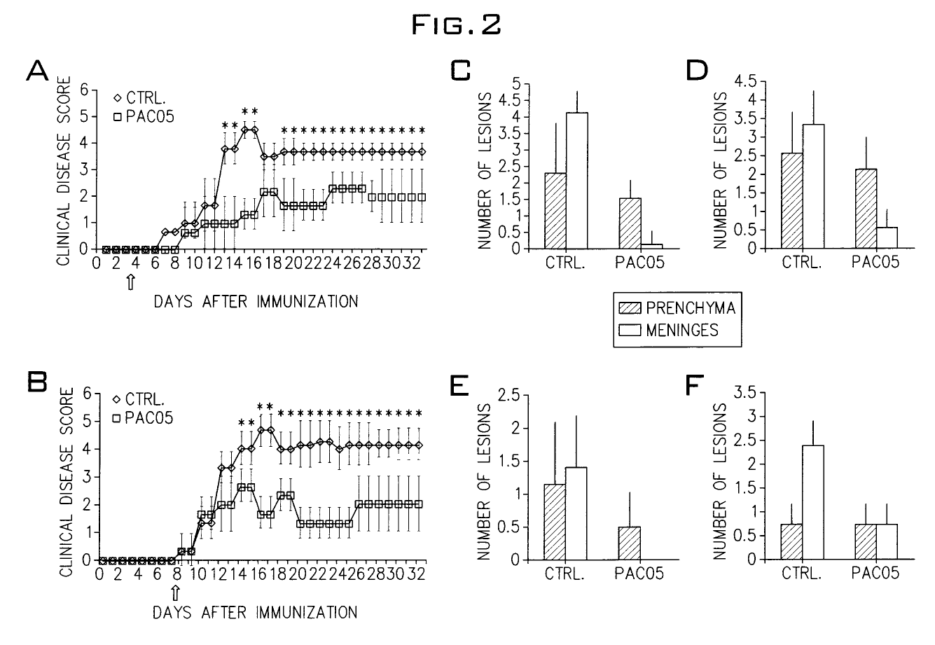

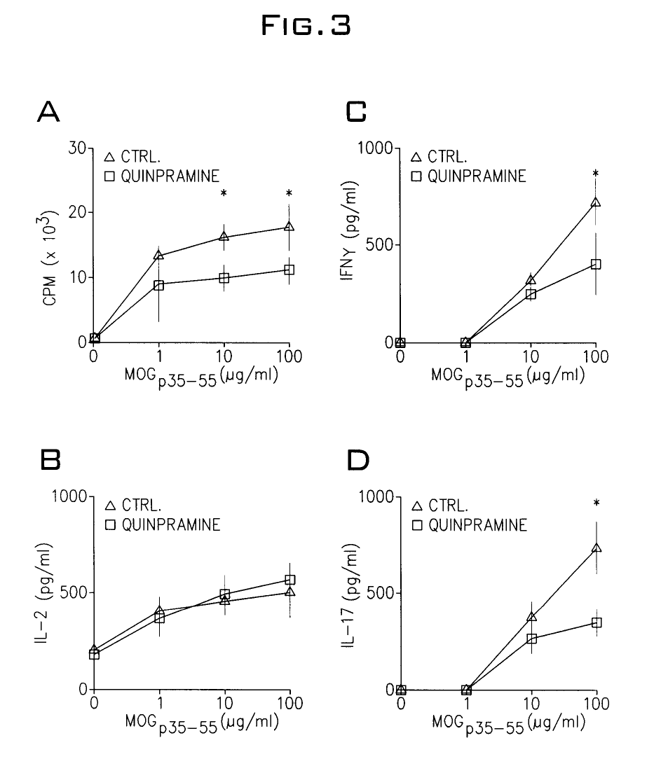

We then probed PAC05's effects on an animal model of multiple sclerosis, experimental autoimmune encephalitis (EAE). The idea was that the assumed effects of PAC05 on lipid rafts might influence signal transduction events on the immunological synapse of T-lymphocytes. We were able to show clear immunosuppressive effects of PAC05 on EAE (see the Example 7 and FIGS. 2 and 3). These data suggest that PAC05 may be a candidate drug for the treatment of multiple sclerosis or other autoimmune diseases.

Examples and figures that serve to illustrate the present invention are given in what follows.

### Example 1

Manufacture of 6-chlor-2-methoxy-acridine-9-yl)-(4-{4-[3-(10,11-dihydro-dibenzo[b,f]azepine-5-yl)-propyl]-piperazine-1-yl}-butyl)-amine.

Na (Oac)₃ BH₃ (171 mg, 0.80 mmol) were added to a solution of 3-(10,11-dihydro-dibenzo[b,f]azepine-5-yl)-propionaldehyde (100 mg, 0.40 mmol) and N-(3-cyanopropyl)-piperazine (122 mg, 0.80 mmol) in CH₂Cl₂ (10 ml). After 16 hours of stirring, the mixture was diluted with CH₂Cl₂ (50 ml) and washed with a saturated solution of NaHCO₃. The organic phase was dried (Na₂SO₄) and evaporated. Purification using flash chromatography of the residue obtained yielded N-[3-(10,11-dihydro-dibenzo[b,f]azepine-5-yl)-propyl]-N′-(3-cyanopropyl)-piperazine (yield: 106 mg, 68%).

N-[3-(10,11-dihydro-dibenzo[b,f]azepine-5-yl)-propyl]-N′-(3-cyanopropyl)-piperazine (60 mg, 0.15 mmol) was dissolved in dried diethylether (Et₂O) (5 ml) and mixed with LiAlH₄ (1.0 M in Et₂O, 0.15 ml, 0.15 mmol) at 0° C. After a reaction time of 3 hours, 3 drops of 0.5 N NaOH were added and the mixture was filtered via silica gel (Celite®, 521 AW) and MgSO₄. Evaporation of the solvent yielded 48 mg N-[3-(10,11-dihydro-dibenzo[b,f]azepine-5-yl)-propyl]-N′-(3-aminopropyl)-piperazine, which was dissolved during mild heating in 1.5 g of phenol. Addition of 6,0-dichloro-2-methoxy-acridine (70 mg, 0.25 mmol) was followed by 1 hour of stirring at 100° C. Subsequent addition of aqueous NaOH, extraction with EtOAc and vaporization of the organic phase yielded 6-chlor-2-methoxy-acridine-9-yl)-(4-{4-[3-(10,11-dihydro-dibenzo[b,f]azepine-5-yl)-propyl]-piperazine-1-yl}-butyl)-amine, which was purified via flash chromatography, in a yield of 21%.

### Example 2

Isolation and purification of a polyclonal chicken anti-mouse prion protein antibody.

Immunoglobin Isolation:

Eggs from chickens immunized with recombinant mouse prion proteins (recMoPrP) were collected. The egg yolks were diluted at a 1:5 ratio in cold 20 mM sodium acetate solution, pH 5.2, and kept overnight at 4° C. After centrifuging to remove insoluble material at 20,000×g for 20 minutes, the immunoglobin was precipitated by addition of 20% (w/v) of (NH₄)SO₄ and centrifuged after 2 hours at 4° C. at 20,000×g for 20 minutes. The palletized immunoglobin was dissolved in a solution of 20 mM Tris pH 8, 150 mM NaCl, 0.1% tween-20, 1 mM EDTA.

Purification:

Recombinant mouse prion protein (recMoPrP) was dissolved in a suspension of NHS-activated sepharose from Amersham Pharmacia, pre-washed according to the directives of the manufacturer's protocol, in a cold (4° C.) solution of 50 mM of NaHCO₃, pH 8.3, 1% triton X-100, 20% DMSO at 3 mg of protein per ml of resin. This suspension was stirred overnight at 4° C. Free NHS groups were then blocked with 50 mM glycine for 1 hour at room temperature. The resin was then washed consecutively with solutions of 50 mM Tris ph 9, 50 mM glycine pH 3, and finally 20 mM Tris pH 8, 150 NaCl, 0.2% triton X100, 0.2% Tween-20, 2 mH EDTA. In this buffer, immobilized recombinant mouse prion protein (recMoPrP) was mixed with anti-mouse prion protein immunoglobin (approximately 200 mg of immunoglobin per mg of mouse prion protein) and stirred overnight at 4° C. The resin was then collected and thoroughly washed, and then elutriated with a solution of 100 mM glycine pH 3, 1 M NaCl, 1% triton X-100, whereby the pH was then immediately set to 8.

The purified anti-mouse prion protein immunoglobin was then tagged with EZ-Link™ maleimide activated horseradish peroxidase (HRP) from Pierce Chemical, Rockford, Ill., according to the directives of the manufacturer's protocol.

This antibody merely recognizes the non- and monoglycosiliated form of the prion protein, that corresponds to the misfolded prion protein, but not the diglycosliated form.

### Example 3

Cell culture trials with compounds of formulas (12), (13) and (14).

Mouse neuroblastoma cells (N2 a) were infected with mouse-adapted scrapie prions and subcloned (Bosque, P. J. and S. B. Prusiner (2000) “Cultured cell sublines highly susceptible to prion infection”-J Virol 74(9): 4377-86) and cultivated. Confluent 10-cm cell culture dishes were split, a drop with 2-5*10⁴ of the mouse neuroblastoma cells (ScN2 a) infected with mouse-adapted scrapie prions was added into a 60-mm cell culture dish and cultivated with 4 ml MEM from Invitrogen of Carlsbad, U.S.A., containing 10% (vol/vol) of fetal calf serum (FCS), penicillin-streptomycin (100 units per ml) and L-glutamine (2 mM) for one week with 0.1 μM, 0.5 μM and 1 μM of compound (12) and (14) and 0.1 μM and 0.5 μM of compound (13). During the week, on every 2^{nd} day, the medium was changed and the appropriate quantity of the tested compound was added to the medium.

After 7 days the cells were washed once with PBS (137 mM NaCl, 2.7 mM KCl, 10 mM Na₂HPO₄ 2 mM KH₂PO₄) and lysated in a lysis buffer (10 mM Tris, ph 8.0, 150 mM NaCl, 0.5% triton X-100, 0.5% deoxycholate). The lysates were then digested with Proteinase K from Merck in Darmstadt (20 μg per ml, 30 minutes, 37° C.) and the reaction was stopped with 2 mM of PMSF (phenylmethylsulfionyl fluoride). Then the digested lysates were centrifuged for 45 minutes at 4° C., 100,000 g, the excess was removed, and the pellets were absorbed into yellow charging buffer (100 mM Tris-Cl pH 6.8, 4% (w/v) SDS, 0.2% (w/v) bromphenol blue, 20% (v/v) glycerol, 200 mM β-mercaptoethanol). The samples are separated via an SDS gel (4-20%, Biorad) and the prion protein is detected with 0.3 μg per ml of the antibody according to example 2 by western blotting (Enhanced Chemiluminescence (ECL) System from Amersham Pharmacia).

Mouse neuroblastoma cells infected with mouse-adapted scrapie prions served as negative controls, which were not treated with Quinacrin. Cells that were treated with the known anti-prion substance Quinacrin under trial conditions with 1 μm served as the positive controls.

The misfolded form of the prion protein was not destroyed due to treatment with Proteinase K and was detectable in the Western blot.

It was shown that the compounds (12), (13) and (14) demonstrated an elimination of the misfolded prion protein in the infected cells, with compounds (12) and (13) having greater effectiveness than compound (14).

### Example 4

In correspondence to the conditions of Example 3, mouse neuroblastoma cells infected with mouse-adapted scrapie prions were incubated with 0.05 μM, 0.075 μM, 0.1 μM and 0.3 μM of the compound of formula (10), and processed, and the misfolded prion proteins were detected in Western blot.

Mouse neuroblastoma cells infected with mouse-adapted scrapie prions served as negative controls, which were not treated with Quinacrin. Cells that were treated with the known anti-prion substance Quinacrin under trial conditions with 1 μm served as the positive controls. As comparisons, cells that were treated with 0.1 μM and 0.3 μM of Quinacrin were treated.

It was shown that in cell cultures that were incubated with a concentration of 0.1 μM and 0.3 μM of the compound of formula (10), in the lysates, no misfolded prion proteins could be detected, while the cells treated with the identical concentration of Quinacrin still contained misfolded prion proteins. Compound (10) revealed itself to be about 10 times more active than Quinacrin.

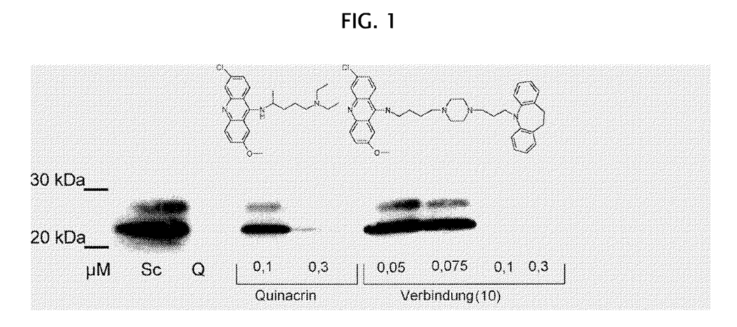

The Western blot depicted in FIG. 1 makes clear the trial results of example 4. FIG. 1 shows Western blots of cell lysates of ScNa2 cells digested by Protease K that show the bands of misfolded prion proteins. Sc stands for the negative check of untreated cells, which shows clear bands of the misfolded prion protein. Q stands for cells treated as positive controls with 1 μm of Quinacrin, which reveal no bands of the misfolded prion protein.

It can be recognized that in cell cultures that were incubated with a concentration of 0.1 μM and 0.3 μM of the compound of formula (10), in the lysates no misfolded prion proteins could be documented, while the cells treated with the identical concentration of Quinacrin still contained misfolded prion proteins.

### Example 5

Filipin Staining.

The cells were applied onto cover slips and treated as in examples 3 and 4 with 0.25 μM of compound (10) and 1 μM of Quinacrin. On the sixth day of the treatment, the cells were washed once with ice-cold PBS (137 mM NaCl, 2.7 mM KCl, 10 mM Na₂HPO₄, 2 mM KH₂PO₄). Then the cells were fixed with 4% (w/v) of paraformaldehyde in PBS for 30 minutes at room temperature. Then the paraformaldehyde solution was removed and the cells were incubated with filipin (50 μg per ml in PBS from Sigma) for 30 minutes at room temperature. The cover slips were washed 4 times with PBS and investigated under the microscope with the appropriate filter sets. The filipin staining detects cellular cholesterol.

It was shown that with the untreated cells (ScN2 a), the cholesterol is predominantly to be found on the cell surface. In the cells treated with compound (10), a redistribution was revealed to intracellular compartments, just as in the cells treated with Quinacrin. This shows the effect of the compounds on cholesterol.

### Example 6

Activity trials in vivo on a mouse model.

The effect of the compound according to formula (10) on a prion infection in vivo was investigated in a mouse model of the prion disease.

Transgene mice (TG 20, Fischer et al., 1996, EMBO Journal, vol. 15, pp. 1255-64) were used, which additionally superexpressed the endogenous mouse protein PrP to endogenous mouse PrP-Gen. These mice have a shortened incubation time for prions of 92±4 days, because, due to the high concentration of prion proteins (PrP^{C}) in the brain, prions multiply especially quickly, so that acting against this accelerated conversion is necessary.

These mice were inoculated three weeks before the start of the trials intracerebrally with 100 μl of a 10⁻⁵ diluted RML scrapie-containing cerebral homogenate (Chandler, Lancet, 1961, vol. 1, pp. 1378-79). Thus at the start of the trials already a positive effect was made difficult, because neurotoxic effects and/or subclinical brain damage could arise.

The compound according to the formula (10) was first dissolved in ethanol, and then, in thistle oil, diluted in an ending concentration of 10 mg per ml. Then the compound was applied via a stomach probe, in Gavage feeding mode, to a group of 11 mice, three times weekly on Monday, Wednesday and Friday, for eight weeks, at a dose of 2 mg per 200 μl per mouse.

A control group of mice that were not treated with the compound according to formula (10), correspondingly died after 92±4 days of prion disease, while the mice that were treated with the compound according to formula (10), died only after 100±5 days.

Thus, we found a lengthening of the survival time by 10%, determined using Student's t test (p<0.0001), under the trial conditions. These conditions come close to the clinical situation in which patients with Creutzfeldt-Jakob disease only seek medical help in the later stages.

### Example 7

Peptides and Antibodies.

Mouse MOG peptide 35-55 (MEVGWYRSPFSRVVHLYRNGK) was synthesized by solid-phase Fmoc chemistry (C—S Bio, USA).

Mice.

Female C57BL/6 mice were purchased from the Jackson Laboratory (Bar Harbor) and bred in a barrier animal facility at UT Southwestern Medical Center. All protocols involving mice handling were approved by the UTSW Institutional Animal Care and Use Committee (IACUC).

Experimental Autoimmune Encephalomyelitis.

EAE was induced in 8-12 weeks old female C57BL/6 mice by immunization with 100 μg of MOGp35-55, which was dissolved in complete Freund's adjuvant (CFA) containing 4 mg ml⁻¹ of heat-killed Mycobacterium tuberculosis H37 Ra (Difco Laboratories). Mice were examined daily for clinical signs of EAE and scored, where: 0, no paralysis; 1, loss of tail tone; 2, hindlimb weakness; 3, hindlimb paralysis; 4, hindlimb and forelimb paralysis; 5, moribund or dead.

For in vivo treatment of experimental animals, PAC05 was dissolved in 100% ethanol (final concentration<2%). PAC05 was than diluted with 100% safflower oil. The final concentration per mouse was 1 mg/100 μl (2% ethanol, 98% safflower oil). Control animals were treated with 100 μl of 2% ethanol and 98% safflower oil alone. Both treatments were administered using 20 mm feeding needles (Popper and Sons, Inc.) on Mondays, Wednesdays, and Fridays.

Histopathology.

Anaesthetized mice were perfused with 20 ml cold PBS. Brains and spinal cords were fixed in 4% (w/v) paraformaldehyde and embedded in paraffin. Sections were stained with haematoxylin and eosin. Selected brain, thoracic and lumbar spinal cord sections were evaluated by an examiner blinded to the treatment status of the animal. Inflammatory foci were counted in meninges and parenchyma.

Proliferation Assays.

For primary proliferation assays, splenocytes were isolated and cultured in vitro with MOGₚ₃₅₋₅₅. Cells were cultured in 96-well microtitre plates at a concentration of 5×10⁶ cells ml⁻¹. Culture medium consisted of RPMI 1640 (Invitrogen) supplemented with L-glutamine (2 mM), sodium pyruvate (1 mM), non-essential amino acids (0.1 mM), penicillin (100 U ml-1), streptomycin (0.1 mg ml-1), 2-mercaptoethanol (5 10-5 M) and 1% (v/v) autologous normal mouse serum. Splenocytes were incubated for 72 h. Cultures were then pulsed for 18 h with 1 μCi per well of [³H]thymidine before harvesting. Cells were then washed and irradiated, and pulsed with MBP_{Ac1-11}. Cells were harvested on glass filters using a Tomtec harvester (Tomtec), and incorporated [methyl-³H]thymidine was measured with a Betaplate counter (PerkinElmer). Results are shown as mean of triplicates±s.e.m.

Cytokines Analysis.

Supernatants from splenocytes cultured in parallel with those cells used in proliferation assays tested were used for cytokine analysis. Supernatants were collected for IL-2 (48 h), IFNγ, IL-17 (both at 72 h), and IL-4 (120 h). Quantitative ELISA was performed using paired mAbs specific for corresponding cytokines per manufacturer's recommendations (BD PharMingen). The results or ELISA are expressed as an average of triplicate wells±SD. A SOFTmax ELISA plate reader and software was used for data analysis (Molecular Devices).

FIG. 2 shows that PAC05 treatment ameliorated the clinical course of actively induced experimental autoimmune encephalomyelitis (EAE), and decreased the numbers of inflammatory foci in the brain and spinal cord. (A) In an EAE “prevention” experiment, PAC05 treatment of female C57BL/6 mice was started on day 2 after active immunization for EAE, and resulted in clinically less severe disease than treatment with vehicle alone (Ctrl.). (B) In EAE “treatment” experiments, PAC05 therapy was initiated at the time of clinical onset of EAE. Mice treated with PAC05 had a substantially more benign clinical disease course than animals treated with vehicle alone. EAE data are shown as mean clinical score±standard deviation. Initiation of in vivo PAC05 therapy is indicated by a filled grey arrow. (C—F) PAC05 therapy started at the time of disease onset (“prevention”) of female C57BL/6 mice in which EAE had been actively induced with MOGₚ₃₅₋₅₅ resulted in a decreased number of parenchymal and meningeal inflammatory foci in the brain (C) and spinal cord (D). When PAC05 therapy was initiated at onset of disease (“treatment”), a reduced number of inflammatory lesions was also detected in the brain (E) and spinal cord (F) of experimental animals. Five comparative brain and spinal cord sections per animal were stained with hematoxylin-eosin (H&E), and inflammatory foci were counted in blinded fashion by light microscopy (magnification ×40). Standard deviations are shown.

FIG. 3 shows the effects of PAC05 therapy on antigen-specific T cell proliferation and on cytokine expression of antigen-specific splenocytes. (A) Splenocytes from C57BL/6 mice immunized with MOGₚ₃₅₋₅₅ and treated with PAC05 or vehicle only from the time of induction of experimental autoimmune encephalomyelitis (EAE) were brought into single cell suspension on day 10 after immunization. Proliferation to MOGₚ₃₅₋₅₅ re-stimulation was determined by [³H] thymidine incorporation. Splenocytes of PAC05-treated mice showed significantly decrease antigen-specific proliferation than control (Ctrl.) animals. (B) Interleukin (IL)-2, (C) interferon gamma (IFNγ), and (D) IL-17 expression was measured by enzyme-linked immunosorbent assay (ELISA). PAC05 therapy resulted in decreased expression of all of these cytokines, except IL-2, compared to control experimental animals.

### Example 8

Animal trial of PAC05 in treatment of acute inflammatory neuropathies.

Acute inflammatory neuropathies including the prototypic Guillain-Barré syndrome (GBS) are clinically defined by rapidly progressive flaccid paresis and are considered to result from a misdirected autoimmune reaction against components of the peripheral nervous system. See, Griffin J W, Sheikh K: The Guillain-Barré Syndromes. In Peripheral Neuropathy, edn 4th ed. Edited by Dyck P J, Thomas P K: Elsevier Saunders; 2005:2197-2220. vol 2. Despite the availability of intensive care and disease modifying treatments i.e. plasmapheresis and intravenous immunoglobulins inflammatory neuropathies remain disorders with significant morbidity and mortality. See, Hughes R A, Cornblath D R: Guillain-Barre syndrome. Lancet 2005, 366:1653-1666. Experimental autoimmune neuritis (EAN) can be elicited by immunization of susceptible rodent strains with peripheral nerve components, serves as animal model of inflammatory neuropathies and multiple treatment approaches have been tested in EAN models. See, Meyer zu Horste G, Hartung H P, Kieseier B C: From bench to bedside—experimental rationale for immune-specific therapies in the inflamed peripheral nerve. Nat Clin Pract Neurol 2007, 3:198-211. Many of these treatments were only effective when started at the time of immunization, but not at the time of initial disease manifestation, limiting the possible clinical value of such treatments. This experiment was designed to test whether PAC05 would influence disease manifestation in the animal model of GBS.

Methods & Results:

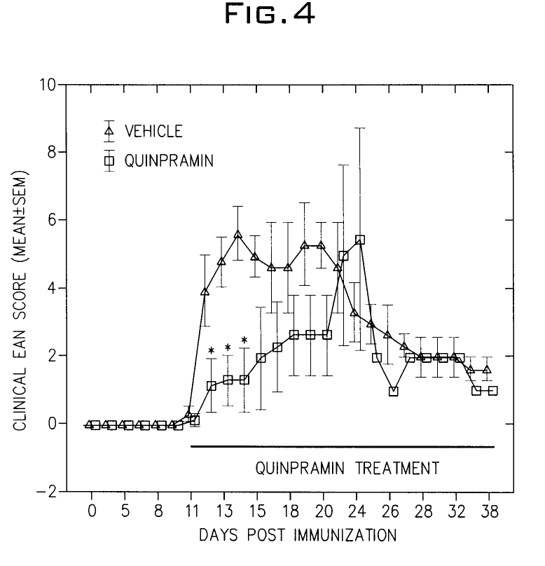

Young adult (8-10 weeks of age) female Lewis rats (n=12) were immunized with subcutaneous injections of bovine peripheral nerve homogenisates (7 mg per animal) emulgated in complete Freund's adjuvant (CFA). All animals developed clinically obvious EAN with acute flaccid paresis of their hind limbs and EAN severity was monitored daily using the well established EAN score (0=no symptoms; 10=death from EAN). Initial disease symptoms were observed from day 10 after immunization onwards. Results are reported in FIG. 4 (Note: PAC05 is identified as “Quinpramin in FIG. 4).

From the time of first clinical symptoms onwards a group of randomly chosen animals (n=6) received oral treatment with PAC05 dissolved in 3% ethanol and 97% safflower oil three times per week using force feeding tubes until the final time point of the experiment (day 38 post immunization). The PAC05 dosage was adjusted to 1 mg/kg body weight at each treatment, thus resulting in a cumulative dosage of 3 mg/kg body weight per week for each animal. A control group (n=6) received vehicle treatment by force feeding only.

In the untreated control group EAN symptoms quickly peaked until day 13 post immunization and remained on an equally high plateau level for 10 days. In contrast PAC05 treated animals did not develop severe paresis, but exhibited marginal clinical EAN symptoms until day 20 post immunization This clinical difference resulted in significantly reduced EAN scores in the PAC05 treated group until day 15 post immunization (FIG. 4). From days 22 to 25 post immunization the PAC05 treated group reached a delayed disease peak of comparable intensity but greatly reduced duration of three days compared to the control group. EAN manifestations slowly declined in both groups, although a slight residual clinical deficit remained in most animals. No second bout or chronic progression of the EAN symptoms was noted until day 38 post immunization

### SUMMARY & CONCLUSION

PAC05 treatment, when initiated at the onset of clinical symptoms, significantly reduced early disease severity in the Lewis rat EAN model and delayed and shortened the disease peak in comparison to vehicle treated controls. PAC05 thus constitutes a promising new candidate drug for the treatment of inflammatory neuropathies.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. A method of treating a human or an animal afflicted with an autoimmune disease comprising the steps of:
providing a compound according to formula (1) and/or an enantiomer, diastereomer and/or a pharmaceutically compatible salt of the compound according to formula (1): 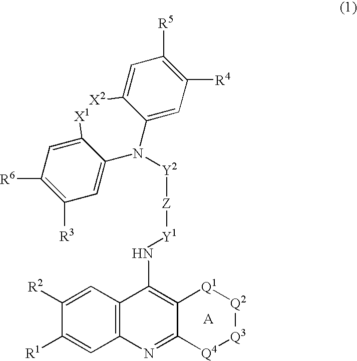
wherein:
A is a six-link, unsaturated or saturated ring;
Q¹, Q², Q³, Q⁴ are each selected, independent of each other, from the group including CH, C-halogen, C—O—(C₁-C₁₀)-alkyl, C—CF₃, C—CN and/or CH₂;
R¹, R², R³, R⁴, R⁵, R⁶, are each selected, independent of each other, from the group including H, Halogen, C₁-C₁₀-alkyl, C₁-C₁₀-alkenyl, C₁-C₁₀-alkinyl, C₁-C₁₀-alkyloxy, CF₃, NH₂, NHR⁹, whereby the radical R⁹, is selected from the group including C₁-C₁₀-alkyl and/or C₁-C₁₀-acyl, NO₂, and/or CN;
X¹, X² each are H, or X¹ and X² jointly form X, whereby:
X is selected from the group including CH₂, CH₂—CH₂, CH═CH, O and/or S;
Y¹, Y² each, independent from each other, are unbranched or branched C₁-C₁₀-alkyl;
Z is a structure element Z1 or Z2 as given above, whereby:
Z1 has the following general formula (2): 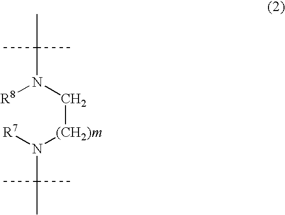
wherein:
m is 1, 2, 3, 4, 5 or 6,
R⁷, R⁸, are selected independently of each other from the group including H, C₁-C₁₀-alkyl, whereby R⁷ and R⁸, if necessary via a CH₂—CH₂ group, form a ring, and/or C₁-C₁₀-acyl; and
Z2 has the following general formula (3): 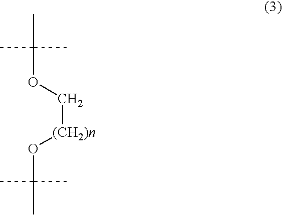
wherein
n is 2, 3, 4, 5 or 6; and
administering the compound according to formula (1) and/or the enantiomer, diastereomer and/or the pharmaceutically compatible salt of the compound according to formula (1) to the human or the animal.

2. The method according to claim 1 wherein the compound according to formula (1) and/or the enantiomer, diastereomer and/or the pharmaceutically compatible salt of the compound according to formula (1) is administered to the human or the animal orally.

3. The method according to claim 2 wherein the compound according to formula (1) and/or the enantiomer, diastereomer and/or the pharmaceutically compatible salt of the compound according to formula (1) is orally administered to the human or to the animal in an amount of from ≧1 mg per day per 75 kg of the human's or the animal's body weight to ≦1000 mg per day per 75 kg of the human's or the animal's body weight.

4. The method according to claim 3 wherein the amount is from ≧10 mg per day per 75 kg of the human's or the animal's body weight to ≦500 mg per day per 75 kg of the human's or the animal's body weight.

5. The method according to claim 3 wherein the amount is from ≧30 mg per day per 75 kg of the human's or the animal's body weight to ≦100 mg per day per 75 kg of the human's or the animal's body weight.

6. The method according to claim 3 wherein the amount is from >30 mg per day per 75 kg of the human's or the animal's body weight to <50 mg per day per 75 kg of the human's or the animal's body weight.

7. The method according to claim 1 wherein the autoimmune disease is selected from the group consisting of: acute disseminated encephalomyelitis (ADEM); Addison's disease; ankylosing spondylitis; antiphospholipid antibody syndrome (APS); aplastic anemia; autoimmune dermatitis; autoimmune glomerulonephritis; autoimmune hepatitis; autoimmune neuritis; autoimmune vasculitis; autoimmune Oophoritis; autoimmune hemolytic anemia; chronic fatigue syndrome; coeliac disease; colitis ulcerosa; Crohn's disease; dermatomyositis; diabetes mellitus type 1; gestational pemphigoid; Goodpasture's syndrome; Graves' disease; Guillain-Barré syndrome (GBS); Hashimoto's disease; idiopathic thrombocytopenic purpura; Kawasaki's disease; lupus erythematosus; multiple sclerosis; myasthenia gravis; opsoclonus myoclonus syndrome (OMS); optic neuritis; Ord's thyroiditis; panarteritis nodosa; pemphigus; pernicious anemia; polyarthritis in dogs; polymyositis; primary biliary cirrhosis; progressive systemic sclerosis; progressive aplastic anemia; psoriasis; rheumatoid arthritis; Reiter's syndrome; sclerodermia; Sharp syndrome; Sjögren's syndrome; Takayasu's arteritis; temporal arteritis (also known as “giant cell arteritis”); warm autoimmune hemolytic anemia; and Wegener's granulomatosis.

8. The method according to claim 1, wherein Z1 has the formula (7):

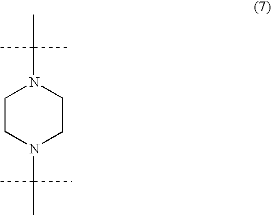

9. The method according to claim 1, wherein the compound according to formula (1) and/or the enantiomer, diastereomer and/or the pharmaceutically compatible salt of the compound according to formula (1) has the formula (9): 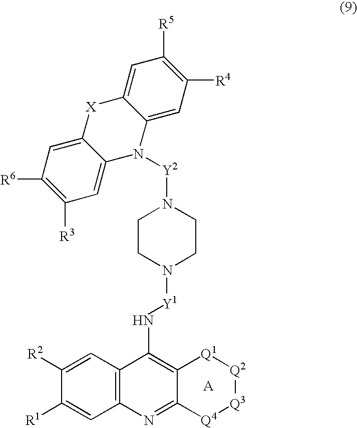
wherein:
A is a six-link, unsaturated or saturated ring;
Q¹, Q², Q³, Q⁴ are each selected, independent of each other, from the group including CH, C-halogen, C—O—(C₁-C₁₀)-alkyl, C—CF₃, C—CN and/or CH₂;
R¹, R², R³, R⁴, R⁵, R⁶ are each selected, independent of each other, from the group including H, Halogen, C₁-C₁₀-alkyl, C₁-C₁₀-alkenyl, C₁-C₁₀-alkinyl, C₁-C₁₀-alkyloxy, CF₃, NH₂, NHR⁹, whereby the radical R⁹, is selected from the group including C₁-C₁₀-alkyl and/or C₁-C₁₀-acyl, NO₂, and/or CN;
X is selected from the group including CH₂, CH₂—CH₂, CH═CH, O and/or S; and
Y¹, Y² each, independent from each other, are unbranched or branched C₁-C₁₀-alkyl.

10. The method according to claim 1, wherein:
Q¹, Q³, and Q⁴ each are CH; and Q² is selected from the group including CH and/or C—O—CH₃.

11. The method according to claim 1, wherein:
Q¹, Q², Q³, and Q⁴ each are CH₂.

12. The method according to claim 1, wherein:
Y¹ and Y² independent of each other, are each selected from the group including —(CH₂)ₒ— and/or —CH(CH₃)—(CH₂)ₚ—, whereby:
o is 2, 3, 4, 5 or 6;
p is 2, 3, 4 or 5.

13. The method according to claim 1, wherein:
R¹, R², R³, R⁴, R⁵, and R⁶ are each selected, independent of each other, from the group including H, halogen, selected from the group including F, Cl and/or Br, NH₂, NHR⁹, whereby the radical R⁹, is preferably selected from the group including C₁-C₅-alkyl, preferably selected from the group including C₁-C₅-acyl, NO₂, C₁-C₅-alkyl, preferably selected from the group including methyl, ethyl, isopropyl, and/or tert-butyl, and/or C₁-C₅-alkyloxy, preferably selected from the group including —O-methyl, —O-ethyl, —O-isopropyl and/or —O-tert-butyl.

14. The method according to claim 1, wherein:
X is S or —CH₂—CH₂—.

15. The method according to claim 4, wherein:
Y¹, Y² independent of each other, are each selected from the group including —(CH₂)ₒ— and/or —CH(CH₃)—(CH₂)ₚ—, whereby:
o is 2, 3, 4, 5 or 6;
p is 2, 3, 4 or 5.

16. The method according to claim 4, wherein:
Y¹, Y² independent of each other, are —(CH₂)ₒ—, whereby:
o is 3, 4 or 5.

17. The method according to claim 10, wherein:
R¹, R², R³, R⁴, R⁵, and R⁶ are each selected, independent of each other, from the group including H and/or Cl.

18. A method of treating a human or an animal afflicted with an autoimmune disease comprising the steps of:
providing a compound according to formula (10) and/or an enantiomer, diastereomer and/or a pharmaceutically compatible salt of the compound according to formula (10): 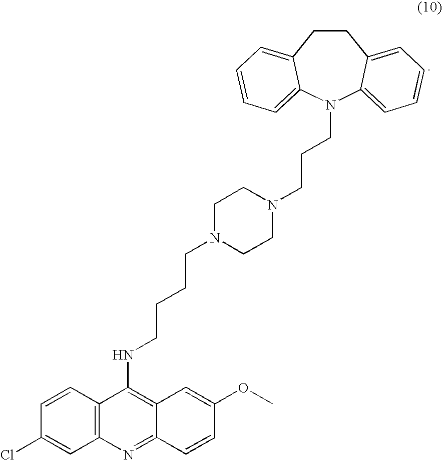
administering the compound according to formula (10) and/or the enantiomer, diastereomer and/or the pharmaceutically compatible salt of the compound according to formula (10) to the human or the animal.

19. The method according to claim 18 wherein the compound according to formula (10) and/or the enantiomer, diastereomer and/or the pharmaceutically compatible salt of the compound according to formula (10) is administered to the human or the animal orally.

20. The method according to claim 19 wherein the compound according to formula (10) and/or the enantiomer, diastereomer and/or the pharmaceutically compatible salt of the compound according to formula (10) is orally administered to the human or to the animal in an amount of from ≧1 mg per day per 75 kg of the human's or the animal's body weight to ≦1000 mg per day per 75 kg of the human's or the animal's body weight.

21. The method according to claim 20 wherein the amount is from ≧10 mg per day per 75 kg of the human's or the animal's body weight to ≦500 mg per day per 75 kg of the human's or the animal's body weight.

22. The method according to claim 20 wherein the amount is from ≧30 mg per day per 75 kg of the human's or the animal's body weight to ≦100 mg per day per 75 kg of the human's or the animal's body weight.

23. The method according to claim 20 wherein the amount is from >30 mg per day per 75 kg of the human's or the animal's body weight to <50 mg per day per 75 kg of the human's or the animal's body weight.

24. The method according to claim 18 wherein the autoimmune disease is selected from the group consisting of: acute disseminated encephalomyelitis (ADEM); Addison's disease; ankylosing spondylitis; antiphospholipid antibody syndrome (APS); aplastic anemia; autoimmune dermatitis; autoimmune glomerulonephritis; autoimmune hepatitis; autoimmune neuritis; autoimmune vasculitis; autoimmune Oophoritis; autoimmune hemolytic anemia; chronic fatigue syndrome; coeliac disease; colitis ulcerosa; Crohn's disease; dermatomyositis; diabetes mellitus type 1; gestational pemphigoid; Goodpasture's syndrome; Graves' disease; Guillain-Barré syndrome (GBS); Hashimoto's disease; idiopathic thrombocytopenic purpura; Kawasaki's disease; lupus erythematosus; multiple sclerosis; myasthenia gravis; opsoclonus myoclonus syndrome (OMS); optic neuritis; Ord's thyroiditis; panarteritis nodosa; pemphigus; pernicious anemia; polyarthritis in dogs; polymyositis; primary biliary cirrhosis; progressive systemic sclerosis; progressive aplastic anemia; psoriasis; rheumatoid arthritis; Reiter's syndrome; sclerodermia; Sharp syndrome; Sjögren's syndrome; Takayasu's arteritis; temporal arteritis (also known as “giant cell arteritis”); warm autoimmune hemolytic anemia;
and Wegener's granulomatosis.

25. The method according to claim 18 wherein the autoimmune disease is multiple sclerosis.
